(19) United States
(12) Patent Application Publication
(10) Pub. No.: US 2025/0230521 A1
(43) Pub. Date: Jul. 17, 2025

(54) Microbial Platform for Rare Earth Bioaccumulation

(71) Applicants: The Regents of the University of California, Oakland, CA (US); San Jose State University Research Foundation, San Jose, CA (US)

(72) Inventors: Nathan Michael Good, Berkeley, CA (US); N. Cecilia Martinez-Gomez, Berkeley, CA (US); Elizabeth Skovran, San Jose, CA (US)

(73) Assignees: The Regents of the University of California, Oakland, CA (US); San Jose State University Research Foundation, San Jose, CA (US)

(21) Appl. No.: 19/059,142

(22) Filed: Feb. 20, 2025

Related U.S. Application Data
(63) Continuation of application No. PCT/US23/72966, filed on Aug. 27, 2023.
(60) Provisional application No. 63/402,874, filed on Aug. 31, 2022.

(51) Int. Cl. C22B 3/18; C12N 1/20; C12R 1/01; C22B 7/00; C22B 59/00

(52) U.S. Cl. CPC C22B 3/18; C12N 1/20; C22B 7/006; C22B 59/00; C12R 2001/01

(57) ABSTRACT

A microbial platform for rare earth element bioaccumulation comprises a bacterial culture, a medium comprising methanol, inorganic phosphate, and a swarf pulp, wherein the platform is selective for bioaccumulation of a rare earth element.

### CROSS-REFERENCES TO RELATED APPLICATIONS

This application is a continuation of PCT/US23/72966, filed: Aug. 27, 2023, which claims priority to U.S. Provisional Application No. 63/402,874; filed: Aug. 31, 2022, the disclosures of which are hereby incorporated by reference in its entirety for all purposes.

### INTRODUCTION

Global demand for rare earth elements (REEs) is at an all-time high and is steadily increasing. REEs, composed of the lanthanides (Lns), and scandium, and yttrium, are critical metals for modern clean energy, communication, advanced transportation, and consumer technologies [1,6,24]. China maintains its status as the world's foremost producer of REEs, generating heavy reliance on a single market that can easily jeopardize REE supply and compromise national security. Simultaneously, a growing understanding of the disastrous environmental impacts of REE mining and production has led to decreased output of REEs by China [13], and an expansion of new mining and production operations worldwide. The development of technologies for domestic REE reuse and recycling has garnered interest as a means of moving towards independence from foreign importation.

Microbiological REE leaching and extraction methods offer promising alternatives to current state-of-the-art methods (hydrometallurgical, pyrometallurgical, and electrometallurgical approaches) that produce large quantities of sludge, acidic wastewater, atmospheric pollution, and radioactive tailings [7,8,13,22]. In particular, microbial bioaccumulation and biomineralization is cost effective and highly efficient for dilute, low-grade, and potentially dangerous REE waste streams [3,18,19]. Bioaccumulation and biomineralization of REEs in bacteria was first shown for the model methylotroph Methylobacterium (also known as Methylorubrum) extorquens AM1 [14]. Methylotrophic bacteria, organisms that thrive on one-carbon compounds such as methane and methanol, provide an attractive new approach for REE bioleaching and extraction due to their natural ability to acquire Ln from the surrounding environment [9,17]. This includes soluble and insoluble low-grade REE sources such as electronic waste (E-waste). Mesophilic methylotrophs have dedicated systems for acquisition, uptake, and intracellular storage of Lns as polyphosphate granules in lanthasome compartments, making them effective agents of bioleaching and bioaccumulation without the need for high acidity or temperature. REE use by mesophilic methylotrophs was thought to be restricted to the light Ln, but recently, a genetic variant of M. extorquens AM1 was isolated and characterized that can transport, store, and grow using the heavy Ln, gadolinium [5]. Detailed genetic and Ln uptake studies indicate the likely possibility of an additional system dedicated to heavy Lns. Thus, methylotrophs may already possess the biological means to separate light and heavy Lns, and have the potential to be engineered for uptake of specific Ln species from mixed, low-grade feedstocks. Further, developing strains resistant to metal toxicity will be vitally important in order to recover REEs from complex, low-grade sources like E-waste. E-waste contains many heavy metals that can generate toxicity, including cadmium, cobalt, manganese, mercury, lead, tungsten, and tellurium.

### SUMMARY OF THE INVENTION

The invention provides a bacterial platform that efficiently and selectively bioaccumulates and recovers rare earths elements (REEs). The invention provides optimization of growth medium conditions for leaching and strain engineering (e.g., generation of genetic variants) to enhance REE bioaccumulation using Methylorubrum/Methylobacterium extorquens, particularly but not exclusively strain AM1. REEs include scandium, yttrium, lanthanum, cerium, praseodymium, neodymium, samarium, europium, gadolinium, terbium, dysprosium, holmium, erbium, thulium, ytterbium, and lutetium. Growth medium amended with REEs, including neodymium magnet swarf (NdFeB magnet), REE salts, REE oxides, REE ores (i.e. monazite, bastnäsite), REE crystals, and electronic waste (cell phone, computer hard drive, speaker, etc.). The invention provides high-yield microbial growth and bioaccumulation with greater than 10% REE source pulp density. The invention provides bioleaching enhancement mediated by REE-bioligand production. The invention provides bioaccumulation enhancement by manipulation of pathways that are independent of carbon metabolism, including phosphate metabolism and REE deregulation of transport. The invention provides construction of a strain of M. extorquens AM1 that is resistant to >5% electronic waste. The invention provides bioaccumulation of REEs in mineral form by M. extorquens AM1. Recovery of pure REE after filtration, cell breakage and organic precipitation.

In an aspect the invention provides a microbial platform for rare earth element (REE) bioaccumulation comprising: a culture of M. extorquens AM1 and a medium comprising: methanol at 10-200 mM, inorganic phosphate at 3-10 mM, and a magnet swarf pulp at 1-10% (w/v) and comprising the REE, wherein the platform has a selective, at least a 2-fold bioconcentration of the REE in the culture.

In embodiments:

the methanol is at 0.01-1%, or about 50 mM.

the inorganic phosphate is at 3.6-7.3 mM, or about 7 mM.

the swarf pulp is 1-5% (w/v), or about 1% (w/v).

the platform has a selective, at least a 3.6-fold bioconcentration of the REE in the culture.

the culture is engineered to overexpress LCC (lanthanide chelation cluster, e.g. META1_4129 through META1_4138) in trans, particularly wherein the platform provides increased Nd bioaccumulation during growth with the insoluble sources Nd₂O₃ (such as 2.3-fold) and magnet swarf (such as 3.2-fold), particularly wherein NdFeB magnet swarf also contains significant amounts of the light Ln Pr and the heavy Ln Dy, and bioaccumulation of these Lns is increased (e.g. on average by 3.5-fold) with expression of the LCC in trans.

the swarf comprises a low-grade REE source, that is ores monazite or bastnäsite, or bastnäsite crystal.

the swarf comprises toxic electronic waste, that is post-consumer electronic waste, like smart phone E-waste, comprising iron, boron, copper, tellurium, manganese, mercury, lead, tungsten, lithium, neodymium, nickel, boron and/or cobalt.

the culture achieves growth with 5% blended cellular phone pulp density.

the M. extorquens AM1 comprises a disrupted oxalate/formate antiporter, oxlT (META1_2757), particularly wherein the oxlT is disrupted by genetic change which disrupts function or expression of the antiporter, wherein the change maybe a natural mutation, or edit, e.g. cre-lox or CRISPR, particularly wherein addition of an effective amount of formate (preferably titrated for optimization, e.g. 2 mM) is used to increase E-waste resistance of the oxlT mutant.

the M. extorquens AM1 comprises a strain having reduced exopolyphosphatase activity to enhance REE bioaccumulation, particularly wherein the strain is selected for reduced exopolyphosphatase activity or comprises a functional deletion or disruption of a ppx gene encoding exopolyphosphatase.

In aspects, the invention provides:

A method for bioaccumulation of REEs comprising operating a platform herein.

A method for bioaccumulation of REEs comprising operating a platform herein, wherein the same swarf batch is sequentially processed for further REE extraction in subsequent bioreactor runs.

A method for bioaccumulation of REEs comprising operating a platform herein wherein the swarf batch is processed in a continuous culture setup to maximize REE extraction from a swarf batch.

A method for bioaccumulation of REEs comprising operating a platform herein wherein bioaccumulation of REE from complex waste streams is effected, including high-grade sources (REE ores, REE oxides, electronic waste) and low-grade sources including REE waste, REE-contaminated waste water and medical waste.

A method for bioaccumulation of REEs comprising operating a platform herein, wherein accumulated REE are purified for commercial or industrial-scale use/reuse in products.

A method for bioaccumulation of REEs comprising operating a platform herein, wherein bioremediation of REE-contaminated water and waste streams is effected.

A method for bioaccumulation of REEs comprising operating a platform herein, wherein accumulated REE are purified for development of lanthanide (sensors)

A method for bioaccumulation of REEs comprising operating a platform herein, for production of biofertilizers, or other agents promoting plant growth.

In aspect, the invention provides a system, platform composition or method as described herein.

The invention encompasses all combinations of the particular embodiments recited herein, as if each combination had been laboriously recited.

### BRIEF DESCRIPTION OF THE DRAWINGS

FIG. 1. M. extorquens AM1 as a platform for Nd recovery through efficient, high-yield bioaccumulation of Nd from complex sources. M. extorquens AM1 is incorporated in a microbial platform for REE recovery due to its innate ability to acquire, transport, and accumulate REE metals into polyphosphate granules.

FIG. 2A-2B. Impact of phosphate and magnet swarf pulp density on strain performance. A, Growth rate of M. extorquens AM1 increases with decreasing inorganic phosphate in the medium. Wild type M. extorquens AM1 was grown in a microplate in Hypho minimal medium with 50 mM methanol and 2 μM NdCl₃. Blue, 14.5 mM phosphate (Pᵢ). Orange, 7.3 mM phosphate (½ Pᵢ). Gray, 3.6 mM phosphate (¼ Pᵢ). Box plots represent the interquartile ranges for 6 biological replicates; standard deviation is shown by error bars B, Yield of M. extorquens AM1 grown on methanol with Nd is influenced by inorganic phosphate in the medium. Box plots represent the averages of 3 biological replicates with interquartile ranges. Error bars are standard deviations.

FIG. 3A-3C. Bioreactor performance with NdFeB magnet swarf. M. extorquens AM1 was grown in 0.75 L bioreactors with reduced phosphate Hypho medium containing 50 mM methanol and Nd magnet swarf. A, growth with 1% swarf. B, growth with 10% swarf pulp density. C, Methanol fed-batch bioreactor cultivation of M. extorquens AM1 with 1% swarf pulp density to obtain higher cell density. Individual data points represent two biological replicates with less than 10% variation.

FIG. 4. Selectivity for REE. M. extorquens AM1 was grown in methanol medium in a benchtop bioreactor with 1% Nd magnet swarf. Cell and supernatant samples were taken once the culture reached early stationary growth phase. Metal concentrations in samples were determined by ICP-MS and normalized to the total metal uptake.

FIG. 5. Strain engineering for increased REE bioaccumulation. Bioaccumulation of REE by M. extorquens AM1 is enhanced by the LCC cluster when using magnet swarf.

FIG. 6. Magnet swarf can be reused for increased REE recovery. Nd bioaccumulation is equally efficient in two consecutive bioreactor process cycles using the same magnet swarf batch (1% pulp density). Plots show the mean of two distinct experiments, each using a single swarf batch for both process runs. Error bars indicate standard deviations.

FIG. 7A-7D. Flexibility for M. extorquens AM1 as a REE recovery platform from low-grade sources. M. extorquens AM1 was grown in methanol medium with and without complex REE sources. A, monazite ore; B, bastnäsite ore; C, bastnäsite crystal. Images are of actual specimens used in growth studies. Ores shown are prior to pulverization. 2 μM LaCl₃ was included as a positive REE source control. Cells were grown in 25×150 mM tubes in a 10 mL culture volume. Aliquots of 200 μL were removed at times shown and the OD 600 was read in a Spectramax M2 plate reader. Background from a no cell control was subtracted from the measurements. D, Growth rates for M. extorquens AM1 with diverse low-grade REE sources. N.G., no growth.

FIG. 8A-F. Toxicity of electronic waste is reduced by oxlT1 deletion. Blended smartphone is a usable Ln source for methanol growth by M. extorquens AM1. A, M. extorquens AM1 was grown in methanol medium with no Lns (open circles), 2 μM LaCl₃ (black squares), or 0.5% (w/v) blended phone with battery (blue triangles). Growth shown as colony forming units per mL of culture. B, increasing blended smartphone to 5% pulp density (w/v) is toxic to M. extorquens AM1. M. extorquens AM1 was unable to grow in methanol medium with smartphone as the only Ln source (blue triangles). Even with the addition of soluble La, 5% smartphone decreased cell viability (black squares). M. extorquens AM1 cell counts did not change significantly over 36 hours when the smartphone was not added (white circles). C, E-waste resistant colonies (EWr for “E-waste resistant M. extorquens AM1”) were isolated by plating M. extorquens AM1 strains on solid methanol medium with 1% blended smartphone. D, The causative mutation for E-waste resistance was identified by genomic sequencing of EWr isolates as a deletion in oxlT. This mutation was reconstructed and allowed methanol growth with 5% smartphone. E, Addition of 2 mM oxalate increased sensitivity to E-waste in the oxlT mutant. F, Addition of 2 mM formate increased E-waste resistance of the oxlT mutant which indicates that media optimization can enhance E-waste resistance further.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### EXAMPLES

M. extorquens AM1 uses the inexpensive single-carbon feedstock methanol, has the innate ability to selectively extract insoluble REEs from the environment by excreting REE-binding ligands (called lanthanophores), and transports them into the cell for use in metabolism and storage in mineral form. We report optimization of growth conditions for bioaccumulation of REEs using M. extorquens AM1 and NdFeB magnet swarf as an E-waste analog. M. extorquens AM1 can efficiently acquire and bioaccumulate REEs from other low-grade sources including REE-rich ores and E-waste. In addition, we demonstrate enhanced REE bioaccumulation by genetically engineering M. extorquens AM1 to overproduce a lanthanophore. Finally, we report a mutation that enables M. extorquens AM1 to be resistant to high pulp densities when using E-waste that bypasses an important bottleneck when scaling up the process.

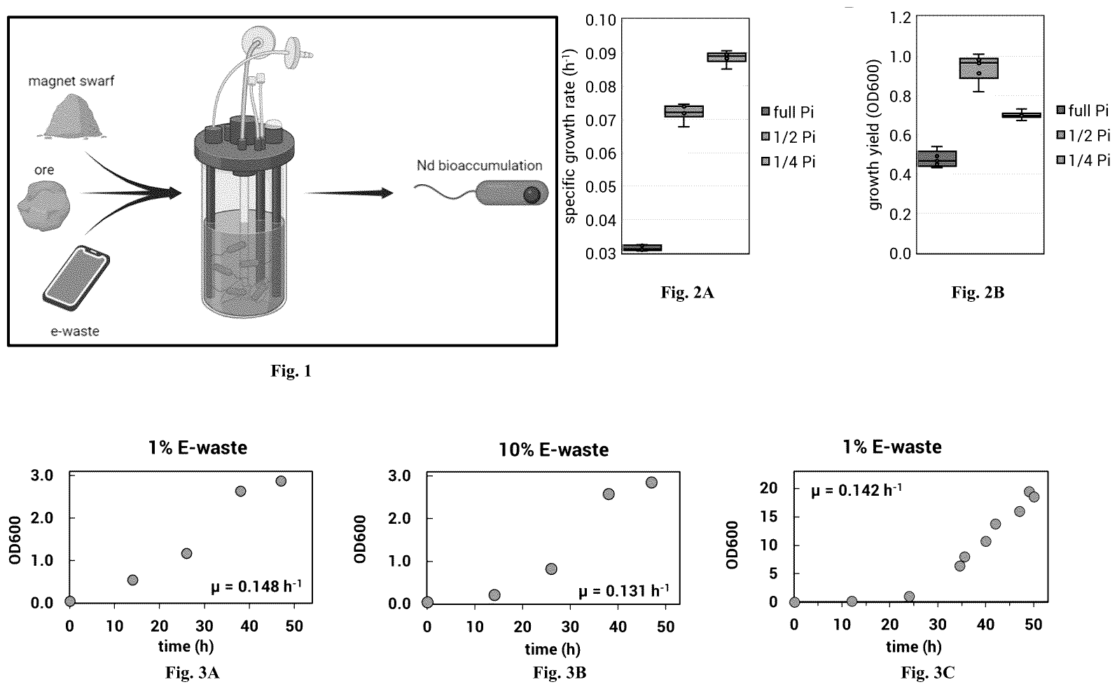

In these examples we demonstrate an optimized method for REE uptake from neodymium magnet swarf (NdFeB) by M. extorquens AM1. We investigated the impacts of methanol substrate concentration, magnet swarf pulp density, and medium composition on Nd bioaccumulation. By optimizing these parameters we have achieved bioaccumulation of 11.5 mg Nd per gram of cell dry weight, or 1.15% dry weight, using up to 10% swarf pulp density. These data demonstrate of the economic viability using M. extorquens AM1 as a platform for biomining and bioaccumulation of REEs from complex feedstocks such as magnet swarf (FIG. 1). In addition, we report efficient growth of M. extorquens AM1 with unprocessed bastnäsite and monazite REE ores, expanding the use of the REE recovery platform to unrefined sources. Finally, we also report the isolation of an E-waste resistant strain of M. extorquens AM1 capable of growth with 5% blended cellular phone pulp density, showing adaptability of the platform for industrial scale applications.

### OPTIMIZATION OF PHOSPHATE IN GROWTH MEDIUM INCREASES GROWTH PERFORMANCE

M. extorquens AM1 biomineralizes REEs as intracellular phosphate granules [14]. Inorganic phosphate is an essential component of defined bacterial growth media, and REE phosphates are poorly soluble. Inorganic phosphate in the growth medium could negatively impact REE solubility and uptake through the formation of REE phosphates, which could hinder REE-dependent methanol growth. To assess the impact of inorganic phosphate on strain performance, we measured the growth rate of M. extorquens AM1 cultured in 48-well microplates in Hypho minimal medium [2] with the standard amount of phosphate (14.5 mM) and compared it to the growth rates of cultures with half the standard amount (7.3 mM) and a quarter of the standard amount (3.6 mM). Growth rate and phosphate concentration exhibited an inverse relationship. The largest relative increase in growth rate (˜2-fold) was observed when inorganic phosphate was decreased to 7.3 mM (FIG. 2A). Growth rate increased an additional ˜0.2-fold when phosphate was reduced to 3.6 mM (FIG. 2A). Reducing phosphate to 7.3 mM resulted in a culture density that was nearly 2-fold higher than culture with the standard phosphate (FIG. 2B). Decreasing phosphate to a quarter of the standard amount increased yield as well, but only by 0.4-fold overall (FIG. 2B).

### BIOREACTOR PERFORMANCE WITH OPTIMIZED GROWTH MEDIUM

M. extorquens AM1 was grown in reduced phosphate (7.3 mM) Hypho medium in a 0.75 L benchtop bioreactor at 30° C. with 50 mM methanol and either 1% or 10% magnet swarf. In the bioreactor, cultures reached maximum densities in ˜40 h (Table 1), representing a 34-50% reduction in growth cycle relative to culturing in shake flasks. Growth rates were significantly increased compared to shake flask cultures: 54% increase with 1% swarf pulp (FIG. 3A) and 26% increase with 10% swarf pulp (FIG. 3B) (growth in flasks not shown). Growth with 1% pulp density produced the highest growth rate and exhibited the shortest cycle time. For these reasons we chose 1% pulp density to investigate bioaccumulation of Nd by M. extorquens AM1.

Our previous work indicated that M. extorquens AM1 accumulated the highest proportion of Lns during stationary phase [14]. Baseline bioaccumulation of cells in stationary phase after growth in the bioreactor with 1% magnet swarf and 50 mM methanol was determined by ICP-MS (Table 1). Bioreactor cultures were grown using reduced phosphate Hypho medium, cell and supernatant samples were collected during exponential, early stationary, and late stationary growth phases, and samples were analyzed for Nd content by ICP-MS. Relative to baseline bioaccumulation levels, bioreactor-grown M. extorquens AM1 accumulated more Nd during each phase of growth. The highest bioaccumulation occurred at early stationary phase (˜26 h), accounting for a nearly 400% increase over baseline and 26% above exponential growth phase samples. Bioaccumulation, normalized to dry weight, decreased by 38% into late stationary phase (50 h), though the culture density only decreased slightly (Table 1).

After successful culturing and Nd bioaccumulation in the methanol-batch bioreactor, we tested M. extorquens AM1 in methanol fed-batch conditions in the bioreactor to scale up culture density and total Nd yield. A culture density of 20 OD was achieved with a reduction in growth rate, corresponding to a ˜50 hr cycle time (FIG. 3C).

### EFFICIENT ACQUISITION AND BIOACCUMULATION OF REE FROM LOW-GRADE SOURCES

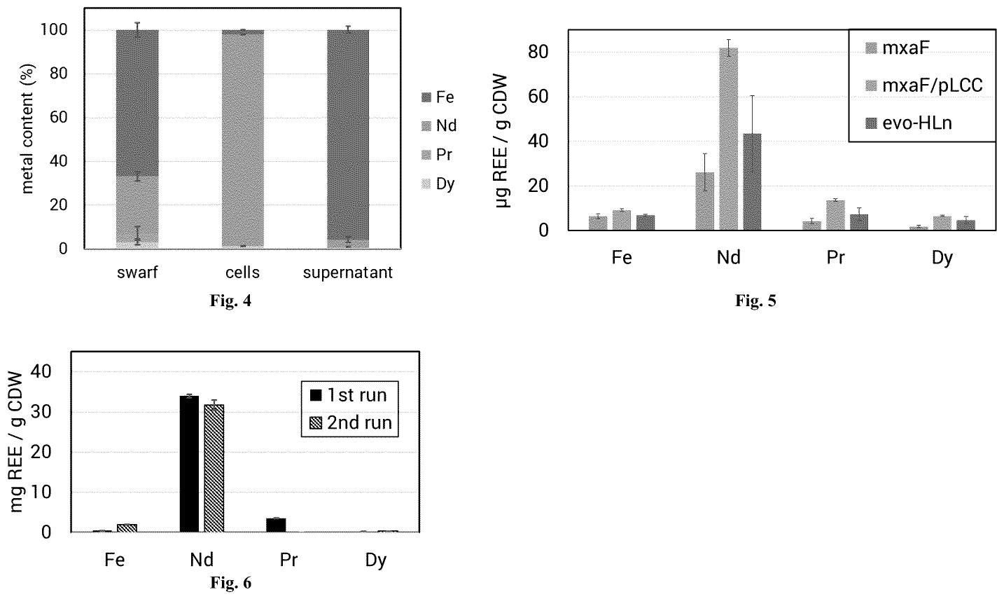

Swarf composition was determined by ICP-MS to be 68.0% Fe, 26.7% Nd, 4.35% Pr, and 3.34% Dy. Fe is the primary metal component of Nd magnet swarf rendering non-selective leaching and uptake mechanisms insufficient for effective REE bioaccumulation. Metal selectivity is crucial for the development of a successful REE recovery platform. Batch bioreactor cultures with 1% magnet swarf were grown maximum culture density with 50 mM methanol (1.8 OD), metal content in cells and supernatant were determined by ICP-MS, and normalized to the total amount of metal accumulated. Of the swarf metal contained in M. extorquens AM1 cells, 96.8% was Nd, and Fe accumulation accounted for only 2.0% of the cell metal content (FIG. 4). Given that cells normally acquire Fe as a micronutrient from the growth medium, the amount of Fe obtained from the magnet swarf is likely much lower. Pr and Dy accounted for 1% combined of metal accumulation (FIG. 4). As REE is leached from the swarf for uptake, we also measured metal content in the culture supernatant. Supernatant metal content consisted of mostly Fe (95.9%), with little REE remaining (Nd, 3.6%; Pr, <1%; Dy, <1%) (FIG. 4). Overall, these data show highly selective 3.6-fold bioconcentration of REE from magnet swarf by M. extorquens AM1.

The identification of the biosynthetic pathway for a REE-chelator has been previously reported (23) and was named LCC (lanthanide chelation cluster), according to the function of its predicted product. Expression of the LCC in trans significantly increased Nd bioaccumulation during growth with the insoluble sources Nd₂O₃ (2.3-fold) and magnet swarf (3.2-fold). NdFeB magnet swarf also contains significant amounts of the light Ln Pr and the heavy Ln Dy, both of which have high technological value. Bioaccumulation of these Lns was increased on average by 3.5-fold with expression of the LCC in trans.

Current extraction yields based on cell density do not extract 100% of the REE present in the magnet swarf. Therefore, we tested if a single magnet swarf batch could be processed for further REE extraction in subsequent bioreactor runs. We confirmed that similar REE recovery yields are obtained from first and second process runs using the same swarf batch. These results open the possibility for implementation of our technology in a continuous culture setup to maximize REE extraction from a swarf batch.

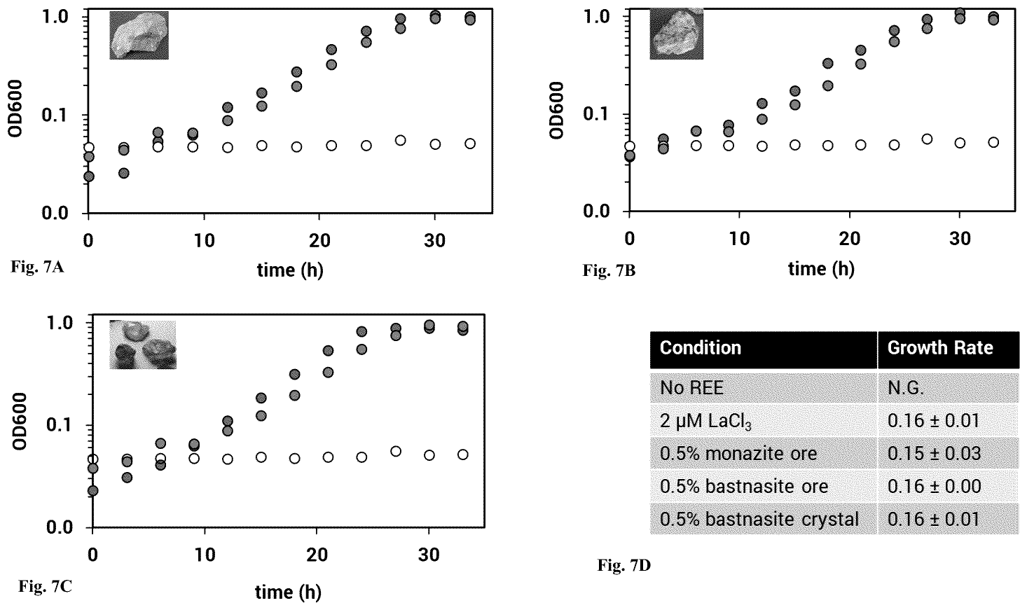

Due to the successful, promising growth and bioaccumulation of Nd from magnet swarf, we tested the ability of M. extorquens AM1 to grow with low-grade REE sources with very low solubility in the growth medium. Monazite ore, for example, contains up to ˜45% REEs, with Ce₂O₃ comprising as much as 17% of the total REEs. However, the REEs in monazite are insoluble as oxide compounds. When challenged to grow on methanol with only the ores monazite or bastnäsite, or bastnäsite crystal, M. extorquens AM1 grew as well as with soluble LaCl₃ (FIG. 7ABC). With each of the REE sources used, the insolubility of the metals and complexity of the source did not significantly impact growth rate or growth yield of the culture (FIG. 7D) showing the flexibility and robustness of M. extorquens AM1 as a biological platform for the bioaccumulation of REE from complex sources.

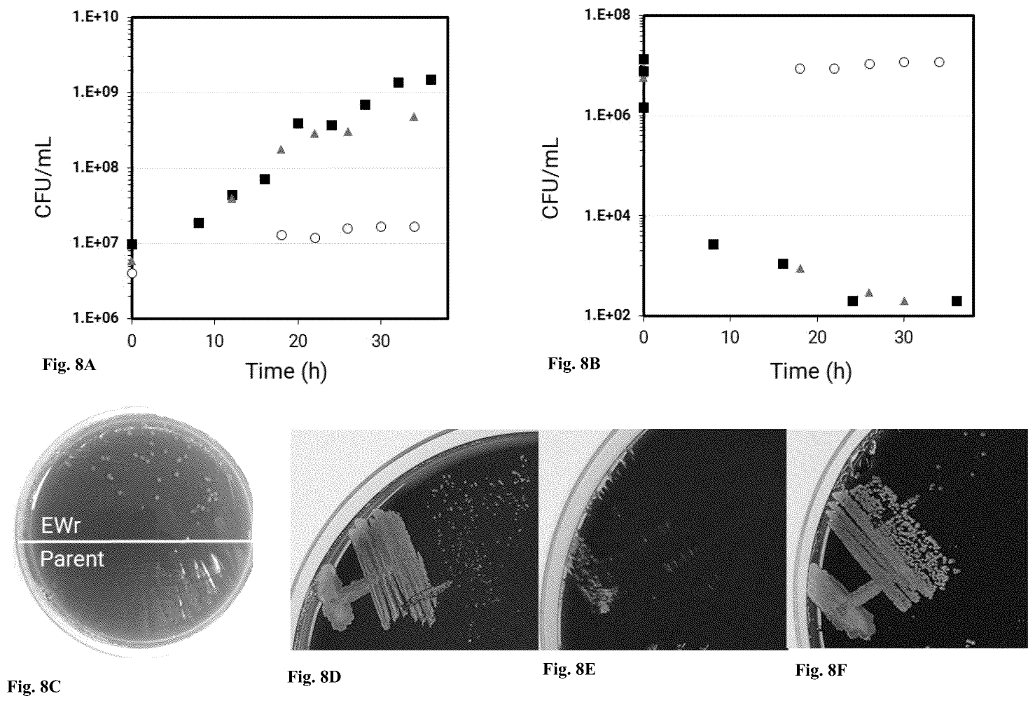

Generation of a strain resistant to toxic electronic waste. Cellular smartphones contain several species of REE, including yttrium, lanthanum, terbium, neodymium, gadolinium and praseodymium, making smartphone E-waste a valuable, untapped source for REE recovery. However, smartphone E-waste poses two major challenges. First, the metals in this E-waste are highly insoluble in oxide form. Further, smartphone batteries contain other metals (e.g. iron, boron, copper, tellurium, manganese, mercury, lead, tungsten, lithium, cobalt), all of which can be toxic. We first tested the feasibility of using smartphone E-waste as a REE source by assessing the capacity of M. extorquens AM1 to grow on methanol. With 0.5% pulp density (w/v) of blended smartphone, M. extorquens AM1 grew as well as with soluble LaCl₃ (FIG. 8A). Next, we assessed the toxicity of smartphone E-waste and saw that increasing pulp density to 5% was enough to eliminate growth even if soluble La was provided as an additional REE source (FIG. 8B). Using 1% pulp density, we were able to isolate colonies that had grown on solid methanol medium, indicating the cells had acquired E-waste resistance (FIG. 8C). The genomes of the E-waste resistant strains were sequenced, and we identified a deletion in the oxalate/formate antiporter, oxlT (META1_2757), as a mutation consistent in all resistant strains. The oxlT deletion was reconstructed, and strains containing this deletion demonstrated E-waste resistance, showing it was the causative mutation (FIG. 8D).

As oxalate/formate antiporters simultaneously import oxalate and excrete fomate, we investigated the effect of adding exogenous formate and oxalate to the growth medium. 2 mM oxalate increased E-waste sensitivity of the oxlT mutant (FIG. 8E) while 2 mM formate further increased resistance (FIG. 8F). These data indicate media optimization can further enhance E-waste resistance.

REEs are stored in intracellular polyphosphate granules in M. extorquens AM1. Depolymerization of polyphosphate is catalyzed by exopolyphosphatase activity. We hypothesized that limiting the cellular capacity for polyphosphate depolymerization could generate higher levels of REE bioaccumulation. A ppx (encoding exopolyphosphatase) deletion strain was generated and assessed for REE bioaccumulation, showing a ˜5.5-fold increase in Nd levels reaching 202 mg Nd/g DW.

TABLE 1
Bioleaching and bioaccumulation of Nd by M. extorquens
AM1 during bioreactor cultivation. Nd content of cell
and supernatant samples were measured by ICP-MS. Fold increase
is relative to the initial bioaccumulation measured for M. extorquens
AM1 before optimization of the growth medium with reduced
phosphate^{ϵ}. Growth phase refers to exponential growth
phase (23-29 h cultivation), early stationary phase
(26-32 h cultivation), and late stationary phase (50
h cultivation).

| growth phase | mg Nd/g CDW | fold increase |
|---|---|---|
| initial^{ϵ} | 3.2± | — |
| exponential | 9.1 ± 3.1 | 2.8 |
| early stationary | 11.5 ± 0.3 | 3.6 |
| late stationary | 7.1 ± 6.4 | 2.2 |

### REFERENCES

[1] K. Binnemans, P. T. Jones, B. Blanpain, T. Van Gerven, Y. Yang, A. Walton, M. Buchert, Recycling of rare earths: a critical review, J. Clean. Prod. 51 (2013) 1-22.

[2] N. F. Delaney, et al, Development of an optimized medium, strain and high-throughput culturing methods for Methylobacterium extorquens, PLOS One. 8 (2013) e62957.

[3] V. Diniz, B. Volesky, Biosorption of La, Eu and Yb using Sargassum biomass, Water Res. 39 (2005) 239-247.

[4] D. H. Figurski, D. R. Helinski, Replication of an origin-containing derivative of plasmid RK2 dependent on a plasmid function provided in trans, Proc. Natl. Acad. Sci. U. S. A. 76 (1979) 1648-1652.

[5] N. M. Good, H. Lee, E. R. Hawker, A. A. Gilad, N. Cecilia Martinez-Gomez, Harnessing methylotrophs as a bacterial platform to reduce adverse effects of the use of the heavy lanthanide gadolinium in magnetic resonance imaging, bioRxiv. (2021) 2021.06.12.448192.

[6] T. E. Graedel, E. M. Harper, N. T. Nassar, B. K. Reck, On the materials basis of modern society, Proc. Natl. Acad. Sci. U. S. A. 112 (2015) 6295-6300.

[7] Y. M. Khawassek, A. A. Eliwa, E. A. Gawad, S. M. Abdo, Recovery of rare earth elements from El-Sela effluent solutions, Journal of Radiation Research and Applied Sciences. 8 (2015) 583-589.

[8] T. Liang, K. Li, L. Wang, State of rare earth elements in different environmental components in mining areas of China, Environ. Monit. Assess. 186 (2014) 1499-1513.

[9] N. C. Martinez-Gomez, H. N. Vu, E. Skovran, Lanthanide Chemistry: From Coordination in Chemical Complexes Shaping Our Technology to Coordination in Enzymes Shaping Bacterial Metabolism, Inorg. Chem. 55 (2016) 10083-10089.

[10] C. J. Marx, M. E. Lidstrom, Broad-host-range cre-lox system for antibiotic marker recycling in gram-negative bacteria, Biotechniques. 33 (2002) 1062-1067.

[11] C. J. Marx, B. N. O'Brien, J. Breezee, M. E. Lidstrom, Novel methylotrophy genes of Methylobacterium extorquens AM1 identified by using transposon mutagenesis including a putative dihydromethanopterin reductase, J. Bacteriol. 185 (2003) 669-673.

[12] D. N. Nunn, M. E. Lidstrom, Isolation and complementation analysis of 10 methanol oxidation mutant classes and identification of the methanol dehydrogenase structural gene of Methylobacterium sp. strain AM1, J. Bacteriol. 166 (1986) 581-590.

[13] Office of Research, Development, Rare earth elements: A review of production, processing, recycling, and associated environmental issues, (2013).

[14] P. Roszczenko-Jasińska, et al, Gene products and processes contributing to lanthanide homeostasis and methanol metabolism in Methylorubrum extorquens AM1, Sci. Rep. 10 (2020) 12663.

[15] K. Schneider, E. Skovran, J. A. Vorholt, Oxalyl-coenzyme A reduction to glyoxylate is the preferred route of oxalate assimilation in Methylobacterium extorquens AM1, J. Bacteriol. 194 (2012) 3144-3155.

[16] R. Simon, U. Priefer, A. Pühler, A Broad Host Range Mobilization System for In Vivo Genetic Engineering: Transposon Mutagenesis in Gram Negative Bacteria, Biotechnology. 1 (1983) 784-791.

[17] E. Skovran, N. C. Martinez-Gomez, Microbiology. Just add lanthanides, Science. 348 (2015) 862-863.

[18] B. Volesky, Detoxification of metal-bearing effluents: biosorption for the next century, Hydrometallurgy. 59 (2001) 203-216.

[19] B. Volesky, Biosorption and me, Water Res. 41 (2007) 4017-4029.

[20] H. N. Vu, et al, Lanthanide-Dependent Regulation of Methanol Oxidation Systems in Methylobacterium extorquens AM1 and Their Contribution to Methanol Growth, J. Bacteriol. 198 (2016) 1250-1259.

[21] Y. Yang, A. Walton, R. Sheridan, K. Güth, R. Gauβ, O. Gutfleisch, M. Buchert, B. M. Steenari, T. Van Gerven, P. T. Jones, K. Binnemans, REE Recovery from End-of-Life NdFeB Permanent Magnet Scrap: A Critical Review, Journal of Sustainable Metallurgy. 3 (2017) 122-149.

[22] L. Zhou, Z. Li, W. Liu, S. Liu, L. Zhang, L. Zhong, X. Luo, H. Liang, Restoration of rare earth mine areas: organic amendments and phytoremediation, Environ. Sci. Pollut. Res. Int. 22 (2015) 17151-17160.

[23] A. M. Zytnick, et al., Identification of a biosynthetic gene cluster encoding a novel lanthanide chelator in Methylorubrum extorquens AM1, bioRxiv. (2022) 2022.01.19.476857.

[24] U.S. Department of Energy. Critical Materials Strategy, 2011.

1. A microbial platform for rare earth element (REE) bioaccumulation comprising:
a culture of M. extorquens AM1; and
a medium comprising:
methanol at 10-200 mM,
inorganic phosphate at 3-10 mM, and
a magnet swarf pulp at 1-10% (w/v) and comprising the REE,
wherein the platform has a selective, at least a 2-fold bioconcentration of the REE in the culture.

2. The platform of claim 1, wherein the methanol is at 0.01-1%.

3. The platform of claim 1, wherein the methanol is at about 50 mM.

4. The platform of claim 1, wherein the inorganic phosphate is at 3.6-7.3 mM.

5. The platform of claim 1, wherein the inorganic phosphate is at about 7 mM.

6. The platform of claim 1, wherein the swarf pulp is 1-5% (w/v).

7. The platform of claim 1, wherein the swarf pulp is about 1% (w/v).

8. The platform of claim 1, wherein the platform has a selective, at least a 3.6-fold bioconcentration of the REE in the culture.

9. The platform of claim 1, wherein the culture is engineered to overexpress LCC (lanthanide chelation cluster, e.g. META1_4129 through META1_4138) in trans.

10. The platform of claim 1, wherein the culture is engineered to overexpress LCC (lanthanide chelation cluster, e.g. META1_4129 through META1_4138) in trans, wherein the platform provides increased Nd bioaccumulation during growth with the insoluble sources Nd₂O₃ (such as 2.3-fold) and magnet swarf (such as 3.2-fold), particularly wherein NdFeB magnet swarf also contains significant amounts of the light Ln Pr and the heavy Ln Dy, and bioaccumulation of these Lns is increased (e.g. on average by 3.5-fold) with expression of the LCC in trans.

11. The platform of claim 1, wherein the swarf comprises a low-grade REE source, that is ores monazite or bastnäsite, or bastnäsite crystal.

12. The platform of claim 1, wherein the swarf comprises toxic electronic waste, that is post-consumer electronic waste, like smart phone E-waste, comprising iron, boron, copper, tellurium, manganese, mercury, lead, tungsten, lithium, neodymium, nickel, boron and/or cobalt.

13. The platform of claim 1, wherein the culture achieves growth with 5% blended cellular phone pulp density.

14. The platform of claim 1, wherein the M. extorquens AM1 comprises a disrupted oxalate/formate antiporter, oxlT (META1_2757).

15. The platform of claim 1, wherein the M. extorquens AM1 comprises a disrupted oxalate/formate antiporter, oxlT (META1_2757), wherein the oxlT is disrupted by genetic change which disrupts function or expression of the antiporter, wherein the change maybe a natural mutation, or edit, e.g. cre-lox or CRISPR.

16. The platform of claim 1, wherein the M. extorquens AM1 comprises a disrupted oxalate/formate antiporter, oxlT (META1_2757), wherein addition of an effective amount of formate (preferably titrated for optimization, e.g. 2 mM) is used to increase E-waste resistance of the oxlT mutant.

17. The platform of claim 1, wherein the M. extorquens AM1 comprises a strain having reduced exopolyphosphatase activity to enhance REE bioaccumulation.

18. The platform of claim 1, wherein the M. extorquens AM1 comprises a strain having reduced exopolyphosphatase activity to enhance REE bioaccumulation, wherein the strain is selected for reduced exopolyphosphatase activity.

19. The platform of claim 1, wherein the M. extorquens AM1 comprises a strain having reduced exopolyphosphatase activity to enhance REE bioaccumulation, wherein the strain comprises a functional deletion or disruption of a ppx gene encoding exopolyphosphatase.

20. A method for bioaccumulation of REEs comprising operating the platform of any claim 1:
wherein the same swarf batch is sequentially processed for further REE extraction in subsequent bioreactor runs;
wherein the swarf batch is processed in a continuous culture setup to maximize REE extraction from a swarf batch;
wherein bioaccumulation of REE from complex waste streams is effected, including high-grade sources (REE ores, REE oxides, electronic waste) and low-grade sources including REE waste, REE-contaminated waste water and medical waste;
wherein accumulated REE are purified for commercial or industrial-scale use/reuse in products;
wherein bioremediation of REE-contaminated water and waste streams is effected;
wherein accumulated REE are purified for development of lanthanide (sensors); or
for production of biofertilizers, or other agents promoting plant growth.
